(19) United States
(12) Patent Application Publication
(10) Pub. No.: US 2024/0242313 A1
(43) Pub. Date: Jul. 18, 2024

(54) DYNAMIC IMAGE PROCESSING APPARATUS, MOVABLE RADIOGRAPHY APPARATUS, DYNAMIC IMAGE PROCESSING SYSTEM, RECORDING MEDIUM, AND DYNAMIC IMAGE PROCESSING METHOD

(71) Applicant: KONICA MINOLTA, INC., Tokyo (JP)

(72) Inventors: Yuuichi NISHIJIMA, Tokyo (JP); Satoshi HASEGAWA, Tokyo (JP)

(73) Assignee: KONICA MINOLTA, INC., Tokyo (JP)

(21) Appl. No.: 18/390,177

(22) Filed: Dec. 20, 2023

(30) Foreign Application Priority Data
Jan. 13, 2023 (JP) 2023-003491

(51) Int. Cl. G06T 5/70; A61B 6/00; G06T 7/00

(52) U.S. Cl. CPC G06T 5/70; A61B 6/4405; A61B 6/5282; G06T 7/0012; G06T 2207/10116; G06T 2207/20092; G06T 2207/30104

(57) ABSTRACT

A dynamic image processing apparatus includes a hardware processor that performs a scattered ray removing process on a dynamic image captured by irradiating a subject with radiation; and acquires at least one imaging condition that is set in capturing the dynamic image. The hardware processor performs the scattered ray removing process on the dynamic image based on at least one of a predetermined condition and the imaging condition that has been acquired.

### CROSS-REFERENCE TO RELATED APPLICATIONS

The entire disclosure of Japanese Patent Application No. 2023-003491, filed on Jan. 13, 2023, including description, claims, drawings and abstract is incorporated herein by reference in its entirety.

### BACKGROUND OF THE INVENTION

### Technical Field

The present invention relates to a dynamic image processing apparatus, a movable radiography apparatus, a dynamic image processing system, a recording medium, and a dynamic image processing method.

### Description of Related Art

In a case where radiography is performed using a grid, a radiographic image in which a scattered ray component is reduced or removed can be obtained. However, a scattered ray component is superimposed on a radiographic image captured without using the grid. For this reason, a technique has been known which performs scattered ray removing process for removing scattered radiation on a radiographic image captured without using a grid in the related art to obtain a high contrast image in which the influence of scattered radiation is reduced.

For example, according to Japanese Unexamined Patent Publication No. 2016-202219 and Japanese Unexamined Patent Publication No. 2014-207958, the scattered ray components are estimated based on the imaging conditions of radiography and the captured image, and the scattered ray components are subtracted from the captured image. Thus, a technology for obtaining a high-contrast image without scattered rays is described.

In addition, a technique for improving the scattered ray removing process speed using inter-frame similarity specific to a moving image in a case in which the radiographic image is the moving image is also known.

For example, Japanese Unexamined Patent Publication No. 2016-063926 focuses on the fact that the calculation time increases in a case where the information on the body thickness distribution, which is one of the parameters necessary for the scattered ray removing process, is calculated for all the frames of the moving image. Furthermore, it is described that after the body thickness distribution is calculated and determined for one frame image, information on the determined body thickness distribution is commonly used for other frame images.

Further, in Japanese Patent No. 7078167, a scattered ray component removal process to be applied to a dynamic image is selected based on order information. Thus, for example, it is described that the processing time for a dynamic image is shortened by not performing removal processing of a scattered ray component depending on the type of analysis.

The influence of the scattered rays on the radiographic image varies depending on the body thickness of the subject, the conditions at the time of radiography, and the like. For this reason, the body thickness of the subject, conditions at the time of radiography (imaging conditions), and the like are considered in the scattered ray removing process. In a case where an image processing apparatus (e.g., a console) that performs image processing such as scattered ray removing process and a radiation emitting apparatus cooperate with each other, the image processing apparatus can acquire imaging conditions from the radiation emitting apparatus.

However, in a system in which the console and the radiation emitting apparatus do not cooperate with each other, the image processing apparatus cannot acquire parameters (actual values) used for actual imaging. Therefore, in scattered ray removing process for a still image of radiation, an estimated value or a preset value stored in an image processing apparatus is used instead of an actual value used for actual imaging.

Furthermore, even in a system in which a radiation emitting apparatus and an image processing apparatus cooperate with each other, a time when parameters (actual values) used for actual imaging are provided from the radiation emitting apparatus to the image processing apparatus is later than the imaging completion timing. It takes a very long processing time to perform the scattered ray removing process for removing the scattered ray component from the radiographic image. For this reason, in order to advance the generation timing of the radiographic image on which the scattered ray removing process has been performed, provision of the actual value from the radiation emitting apparatus may not be waited. In this case, an estimated value or a preset value is used as in the case where the two devices do not cooperate with each other.

In particular, in the case of dynamic imaging, the imaging time is significantly longer than in the case of still image imaging, and furthermore, an enormous amount of time is required for image processes such as the scattered ray removing process. Therefore, it is desirable to start the scattered ray removing process without waiting for the completion of imaging of all the frames.

### SUMMARY OF THE INVENTION

In a case of a still image, the image is simply displayed and observed. For this reason, even when the scattered ray removing process is performed using the estimated value (or the preset value), the deterioration of the image quality at a level which becomes a problem in the observation of the image is not observed.

However, in the case of a dynamic image, not only the dynamic image is displayed and observed, but also various kinds of dynamic analysis may be performed.

In this regard, in a case where the dynamic analysis is performed on the dynamic image subjected to the scattered ray removing process using the estimated value or the preset value, deterioration in image quality which cannot be predicted at the time of simply observing the dynamic image occurs. That is, the image quality degradation caused by the scattered ray removing process using the estimated value or the preset value has not been a problem in the observation of a simple dynamic image. On the other hand, when the dynamic analysis is performed, there is a case where the dynamic analysis cannot be performed well. The inventors of the present invention have found that there is a new problem that cannot be predicted in a case where the display device is used for simply displaying and observing a dynamic image as described above.

The present invention has been made in view of the above-described problem, and an object of the present invention is to provide a dynamic image processing apparatus, a movable radiography apparatus, a dynamic image processing system, a recording medium, and a dynamic image processing method capable of performing favorable dynamic analysis.

To achieve at least one of the abovementioned objects, according to an aspect of the present invention, the dynamic image processing apparatus of the present invention includes:
a hardware processor that
   performs a scattered ray removing process on a dynamic image captured by irradiating a subject with radiation; and
   acquires at least one imaging condition that is set in capturing the dynamic image;
wherein the hardware processor performs the scattered ray removing process on the dynamic image based on at least one of a predetermined condition and the imaging condition that has been acquired.

According to another aspect of the present invention, a dynamic image processing apparatus includes:
a hardware processor that
   performs a scattered ray removing process on a dynamic image captured by irradiating a subject with radiation; and
   acquires at least one imaging condition that is set for capturing the dynamic image; and
a storage that stores a predetermined parameter used in the scattered ray removing process;
wherein the hardware processor performs a scattered ray removing process on the dynamic image based on the parameter to generate a first scattered ray removed image, and the scattered ray removing process on the dynamic image based on at least one imaging condition that has been acquired by the hardware processor to generate a second scattered ray removed image.

According to another aspect of the present invention, a movable radiography apparatus includes the dynamic image processing apparatus according to the present invention.

According to another aspect of the present invention, a dynamic image processing system includes:
a radiation emitting apparatus that emits radiation;
a radiography apparatus that generates a dynamic image based on radiation emitted from the radiation emitting apparatus and transmitted through a subject;
a dynamic image processing apparatus that performs scattered ray removing process on the dynamic image; and
an analysis processing apparatus that performs a dynamic analysis process on the dynamic image on which the scattered ray removing process has been performed,
wherein the dynamic image processing apparatus performs the scattered ray removing process based on at least one of a predetermined condition and an imaging condition that is set for capturing the dynamic image.

According to another aspect of the present invention, a dynamic image processing system includes:
a radiation emitting apparatus that emits radiation;
a radiography apparatus that generates a dynamic image based on radiation emitted from the radiation emitting apparatus and transmitted through a subject;
a dynamic image processing apparatus that performs scattered ray removing process on the dynamic image;

a storage that stores a predetermined parameter used in the scattered ray removing process; and
an analysis processing apparatus that performs a dynamic analysis process on the dynamic image on which the scattered ray removing process has been performed,
wherein the dynamic image processing apparatus performs a first scattered ray removing process on the dynamic image based on the parameter and performs a second scattered ray removing process on the dynamic image based on at least one of a predetermined condition and an imaging condition that is set for capturing the dynamic image.

According to another aspect of the present invention, a computer-readable recording medium storing a program which, when executed by a hardware processor, causes the hardware processor to perform:
an image processing function of performing a scattered ray removing process on a dynamic image captured by irradiating a subject with radiation; and
an acquisition function of acquiring at least one imaging condition that is set for capturing the dynamic image,
wherein, in the image processing function, the hardware processor performs the scattered ray removing process on the dynamic image based on at least one of a predetermined condition and the imaging condition that has been acquired by the acquisition function.

According to another aspect of the present invention, a dynamic image processing method includes:
image processing of performing a scattered ray removing process on a dynamic image that has been captured by irradiating a subject with radiation; and
acquiring at least one imaging condition that is set for capturing the dynamic image,
wherein in the image processing, the scattered ray removing process is performed on the dynamic image based on at least one of a predetermined condition and the imaging condition acquired in the acquiring.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram illustrating an overall configuration example of a dynamic image processing system;

FIG. 2 is a block diagram showing the functional arrangement of the movable radiography apparatus in FIG. 1;

FIG. 3 is an explanatory diagram for explaining imaging conditions acquired by an image processing means at respective timings;

FIG. 4 is a table illustrating an example of parameters included in imaging conditions;

FIG. 5 is a flowchart showing dynamic image processing according to the first technique;

FIG. 6 is a flowchart showing dynamic image processing according to the second technique;

FIG. 7 is a flowchart showing dynamic image processing according to the third technique; and

FIG. 8 is a diagram showing an example of a display screen for displaying an image after scattered ray removing process.

### DETAILED DESCRIPTION

Hereinafter, one or more embodiments of the present invention will be described with reference to the drawings. However, the scope of the invention is not limited to the disclosed embodiments.

### (Configuration of Dynamic Image Processing System 100)

First, a configuration of an embodiment of the present invention will be described.

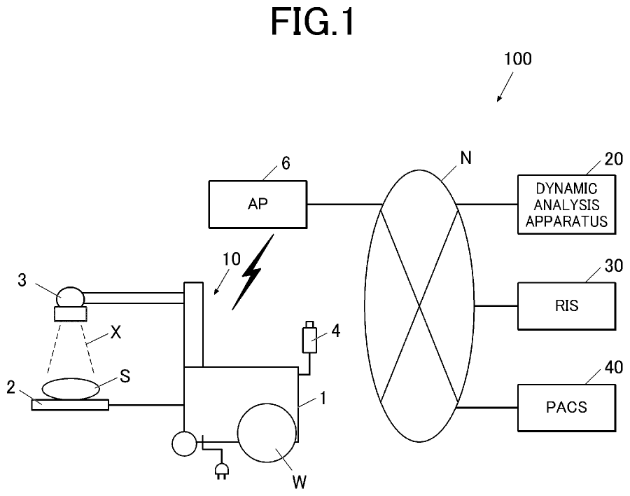

FIG. 1 illustrates an example of the overall configuration of a dynamic image processing system 100 according to the present embodiment. As illustrated in FIG. 1, the dynamic image processing system 100 includes a movable radiography apparatus 10, a dynamic analysis apparatus 20, a radiology information system (RIS) 30, and a picture archiving and communication system (PACS) 40. These devices are connected to each other via a communication network N such as a local area network (LAN) or a wide area network (WAN) so as to transmit and receive data. The movable radiography apparatus 10 is connected to a communication network N via a wireless access point (“AP” in FIG. 1 and the like) 6 of a wireless LAN or a wired LAN cable (not illustrated). A plurality of wireless access points 6 are provided in a medical facility in which the dynamic image processing system 100 is installed.

The respective devices configuring the dynamic image processing system 100 comply with the Digital Image and Communications in Medicine (DICOM) standard, and communication between the devices is performed in accordance with DICOM.

The movable radiography apparatus 10 is, for example, an apparatus for performing radiography of a patient who is difficult to move around in a doctor's office. The movable radiography apparatus 10 includes wheels W on a main body 1, and is configured as a mobile medical cart. Note that the movable radiography apparatus 10 may be a portable apparatus that does not include wheels.

The main body 1 is provided with a storage portion (not illustrated) for storing a flat panel detector (FPD) 2 described below. The housing part is provided with a connector for connecting to the housed FPD 2, and the battery of the housed FPD 2 can be carried while being charged.

The movable radiography apparatus 10 is brought into, for example, an operating room, an intensive care unit (ICU), a patient's room, or the like. Next, radiation is applied from the radiation source 3 in a state in which, for example, the endoscope FPD 2 is inserted between the subject S lying on a bed and the bed or in an insertion slot provided on a surface of a bed (not illustrated) opposite to a surface on which the subject S lies. Accordingly, the movable radiography apparatus 10 performs still imaging or dynamic imaging of the subject S. In the present embodiment, the still imaging refers to acquiring one image of the subject S in response to one imaging operation. Dynamic imaging refers to acquiring a plurality of images of a subject S by repeatedly irradiating the subject S with pulsed radiation such as X-rays at predetermined time intervals (pulse irradiation) or continuously irradiating the subject S with radiation at a low dose rate (continuous irradiation) in response to a single imaging operation. A series of images obtained by the dynamic imaging is referred to as a dynamic image. Each of the images constituting the dynamic image is referred to as a frame image.

Here, the dynamic imaging includes capturing a moving image, but does not include capturing a still image while displaying a moving image. The dynamic image includes a moving image, but does not include an image obtained by capturing a still image while displaying the moving image.

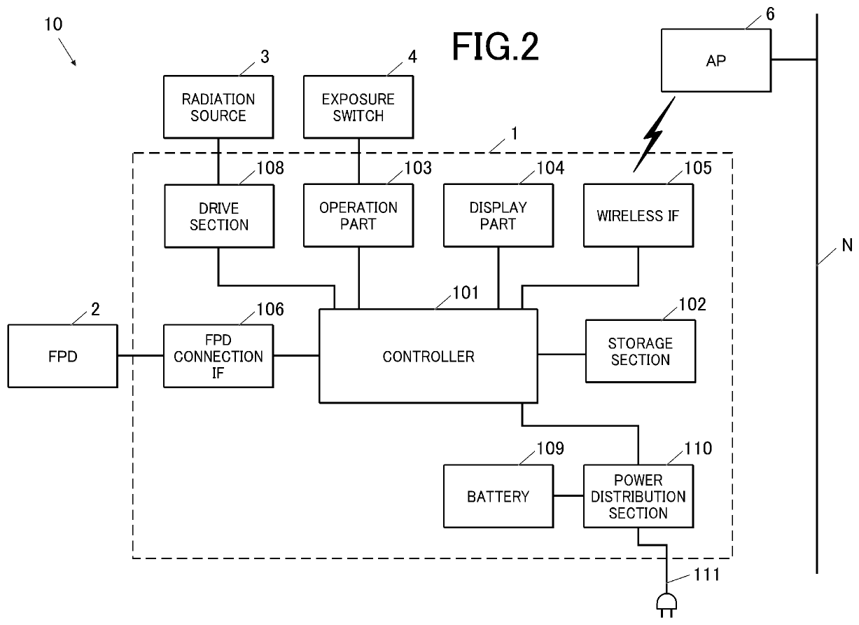

FIG. 2 is a block diagram illustrating the functional configuration of the movable radiography apparatus 10.

As shown in FIG. 2, the movable radiography apparatus 10 is configured to include an FPD 2, a radiation source 3, an exposure switch 4, and the like in addition to the main body 1.

The main body 1 of the movable radiography apparatus 10 has functions as a console and a dynamic image processing apparatus.

As illustrated in FIG. 2, the main body 1 is configured to include an operation part 103, a display part 104, a wireless IF 105, an FPD connection IF 106, a drive section 108, a battery 109, a power distribution section 110, and the like in addition to a controller 101 and a storage section 102.

The controller 101 is configured to include an arithmetic unit such as a central processing unit (CPU), a random access memory (RAM) that provides a work area, and the like (none of which are illustrated). The storage section 102 is a storage means (storage) configured to include a read only memory (ROM) (not illustrated).

The CPU of the controller 101 reads out a system program and various processing programs stored in the storage section 102 and loads them in the RAM in response to an input from the operation part 103 or the like. Then, the CPU of the controller 101 executes various processes in accordance with the loaded program. The controller 101 and the storage section 102 constitute a computer that controls the operation of each unit of the movable radiography apparatus 10 in cooperation with various programs.

For example, in the present embodiment, the controller 101 performs scattered ray removing process on a dynamic image captured by irradiating the subject S with radiation. Furthermore, the controller 101 acquires at least one of setting conditions (also referred to as “imaging conditions”) to be set for capturing a dynamic image.

The controller 101, which will be described in more detail later, performs scattered ray removing process on the dynamic image based on at least one of the “predetermined conditions” and the setting conditions acquired as an acquisition means. The scattered ray removing process for the dynamic image is performed in accordance with a program (a scattered ray removing processing program) stored in the ROM.

Note that in the present embodiment, the “scattered ray removing process” refers to a process for removing a scattered ray component included in an image (dynamic image) (to a level that does not affect image analysis or the like). In the present embodiment, the “scattered ray removing process” includes not only a process for completely removing a scattered ray component but also a process for reducing a scattered ray component.

The storage section 102 which is a storage means in the present embodiment stores a predetermined parameter for the scattered ray removing process.

Here, the “predetermined parameter” is a default value of a parameter applied to the scattered ray removing process and is a preset setting condition (imaging condition). Hereinafter, the “predetermined parameter” is also referred to as a “preset value”.

Order information (information on an examination order) is transmitted from the RIS 30 to the main body 1 of the movable radiography apparatus 10. The order information includes information indicating the content of the order of each imaging operation included in the examination. The controller 101 can specify the content of the order (examination order) and imaging conditions corresponding thereto from the order information.

Specifically, the order information (information on the examination order) includes examination identification information (such as an examination ID), an examination date, and patient information (such as a patient ID, a name, sex, an age, and a hospital room (ward)) on a patient who is the subject S. The order information (information on the examination order)) includes information on each imaging performed in the examination (imaging ID, imaging type indicating distinction between still image imaging and dynamic imaging, type of the analysis process performed by the dynamic analysis apparatus 20 (type of analysis mode, specifically, type or name of the analysis process), requested department, type of emergency or not, and the like).

As described above, in the embodiment, the order information (information of the examination order) on the dynamic imaging from RIS 30 includes information on the type of the dynamic analysis (the type of the analysis mode) performed in the dynamic analysis apparatus to be described later.

The information related to the type of dynamic analysis is information capable of specifying the type of dynamic analysis to be performed on a dynamic image obtained by imaging.

In the present embodiment, in the main body 1 of the movable radiography apparatus 10 which functions as a console and a dynamic image processing apparatus, image processing such as the scattered ray removing process is appropriately performed on an image obtained by dynamic imaging. However, for example, when the scattered ray removing process is not appropriately performed, there is a concern that the subsequent dynamic analysis may not be precisely performed.

Therefore, as described later, when the order (examination order) includes a “predetermined analysis process”, imaging conditions (“final imaging setting value” and “actual value (irradiation actual value)” described later) actually used for capturing of a dynamic image are applied as parameters to the scattered ray removing process. In this case, it takes a little more time than the case of applying a “preset value” which is a default value set in advance and stored in the storage section 102.

Here, the “predetermined analysis process” is, for example, a blood flow analysis process or a ventilation analysis process in the case of a dynamic image obtained by imaging the chest of the subject. In a case of performing a dynamic analysis for acquiring dynamic information on a respiratory organ or a circulatory organ, such as a blood flow analysis process or a ventilation analysis process, it is necessary to finely analyze a slight change in a signal value or the like. For this reason, an appropriate process is required to be performed on a dynamic image to be subjected to dynamic analysis.

Furthermore, the storage section 102 is provided with a temporary storage region for temporarily storage area (e.g., a dynamic image (original image) or a processed image subjected to the scattered ray removing process) waiting to be transmitted to an external device (e.g., the dynamic analysis apparatus 20).

The imaging conditions include, for example, a tube voltage [kV], a tube current [mA], an irradiation time [ms], an exposure dose [mAs], an imaging distance (SID) [cm], grid information (presence or absence of a grid) at the time of imaging, a frame rate, and the type (for example, FPD 2) of a radiation detector.

The storage section 102 stores a table or the like which associates the content of an order (examination order) with imaging conditions corresponding to each order. When order information (information on an examination order) is transmitted from the RIS 30, the controller 101 refers to the storage section 102. Thus, imaging conditions corresponding to the examination order can be acquired.

The operation part 103 is provided with operation buttons, a touch screen in which transparent electrodes are arranged in a lattice shape so as to cover the surface of the display part 104, and the like, and detects operation content by the user. Next, the detection result is output to the controller 101 as operation information. Here, the operation content by the user is a type of the pressed operation button, a contact position operated by a finger or a touch pen, or the like. The content which is operated and input from the operation part 103 and which is output to the controller 101 includes, for example, setting conditions which are set for capturing of a dynamic image. The controller 101 acquires the content output from the operation part 103 as an acquisition means.

Furthermore, an exposure switch 4 with which a user provides instruction for irradiation with the radiation X is connected to the operation part 103.

The exposure switch 4 may be connected to the movable radiography apparatus 10 in a wired or wireless manner so as to be remotely operable. In this way, the user can control the emission of radiation from a place away from the main body 1 of the movable radiography apparatus 10.

The display part 104 is constituted by a monitor such as a liquid crystal display (LCD) or a cathode ray tube (CRT). The display part 104 displays examination order information, a captured image, and the like in accordance with an instruction of a display signal input from the controller 101.

Note that the display part 104 may be connected to the movable radiography apparatus 10 in a wired or wireless manner so as to be capable of performing remote display. In this way, the user can check various kinds of information from a place away from the main body 1 of the movable radiography apparatus 10.

In addition, a sub monitor different from the display part 104 may be connected in a wired or wireless manner.

The wireless IF (wireless communication unit) 104 is wirelessly connected to the wireless access point 6. The wireless IF (wireless communication unit) 104 is an interface for transmitting and receiving (inputting and outputting) data to and from an external apparatus connected to the communication network N via the wireless access point 6. The external device is, for example, the dynamic analysis apparatus 20 or a RIS 30. The wireless IF 104 functions as a detection unit that receives radio waves from the wireless access point 6 and detects the wireless access point 6. In addition, the radio wave intensity of the received radio wave is acquired.

The movable radiography apparatus 10 may include a wired IF (wired communication unit). The wired IF (wired communication unit) is connected to the communication network N through wired communication by inserting a communication cable, and transmits and receives data to and from an external device connected to the communication network N.

In a case where the movable radiography apparatus 10 has a wireless communication unit and a wired communication unit, a connection method with the communication network N can be switched between wired and wireless methods based on a control signal from the controller 101.

The FPD connection IF 106 is an interface for performing data transmission and reception with the FPD 2. The FPD connection IF 106 may transmit and receive data by wireless communication. Furthermore, the FPD connection IF 106 may be one in which a communication cable is inserted to perform data transmission and reception with a FPD 2 by wired communication, or one which supports both of the methods.

Note that if the FPD connection IF 106 supports both methods, the connection method with the FPD 2 can be switched to wired or wireless based on a control signal from the controller 101.

The drive section 108 is a circuit that drives the tube of the radiation source 3. The drive section 108 and the radiation source 3 are connected to each other via a cable or the like.

Upon receiving the control signal from the controller 101, the drive section 108 drives the tube to apply the “predetermined voltage” to the radiation source 3 based on the control signal. The “predetermined voltage” is a voltage corresponding to a preset irradiation condition of radiation. The irradiation conditions of radiation are, for example, conditions related to the irradiation of radiation, such as the imaging type of the dynamic imaging or the still image imaging, a tube voltage, a tube current, an irradiation time, and a current-time product (exposure dose).

The battery 109 is configured to be capable of supplying electric power stored therein to the power distribution section 110 and storing electric power supplied from the power distribution section 110.

The power distribution section 110 has a power source cable 111 provided with a plug at an end thereof. The power distribution section 110 is configured to be able to receive supply of power from an external source by plugging the plug into a nearby outlet. The power distribution section 110 distributes power supplied from the battery 109 or an external source to the various sections of the movable radiography apparatus 10.

The FPD 2 is a radiography apparatus that generates a dynamic image based on radiation X emitted from a radiation source 3 serving as a radiation emitting apparatus and transmitted through the subject S.

The FPD 2 is a cassette-shaped radiation detector. The FPD 2 has a substrate on which pixels arranged in a two-dimensional manner (matrix manner), each including a radiation detection element that receives radiation X to generate an electric charge corresponding to a dose and a switch element that accumulates and discharges an electric charge. The FPD 2 further includes a reading circuit configured to read out the amount of charge discharged from each pixel as a signal value, and a controller configured to generate image data from a plurality of signal values read out by the reading circuit. In addition, the FPD 2 includes a communication unit that transmits various signals to the main body 1 in a wired or wireless manner, a connector into which a cable connected to the main body 1 is inserted, and the like.

Note that FPD 2 may be a so-called indirect type or a so-called direct type. The indirect type FPD 2 incorporates a scintillator or the like, converts the emitted radiation X into light of another wavelength such as visible light by the scintillator, and generates charges corresponding to the converted light. The direct type FPD 2 directly generates a charge from the radiation X without using a scintillator or the like.

The radiation source 3 is a radiation emitting apparatus for emitting radiation X.

The radiation source 3 has, for example, a rotary anode, a filament, and the like (not shown). Next, when a voltage is applied from the drive section 108, the filament emits an electron beam corresponding to the voltage toward the rotating anode, and the rotating anode generates radiation X in a dose corresponding to the intensity of the electron beam.

The dynamic analysis apparatus 20 is an analysis processing apparatus that performs a dynamic analysis process on the dynamic image output from the movable radiography apparatus 10.

In the present embodiment, the dynamic analysis apparatus 20 performs an analysis process (dynamic analysis) on the dynamic image on which the scattered ray removing process has been performed by the controller 101 serving as the dynamic image processing apparatus. Then, the dynamic analysis apparatus 20 transmits the dynamic image and the analysis result to the PACS 40. The dynamic analysis apparatus 20 is capable of performing a plurality of types of analysis processes (dynamic analysis). The dynamic analysis apparatus 20 performs analysis processes (dynamic analysis) of a type specified by the order information out of a plurality of types of analysis processes (dynamic analysis).

For example, in the case of a dynamic image obtained by imaging the chest of a subject, various kinds of the dynamic analysis process including the “predetermined analysis process” such as a blood flow analysis process and a ventilation analysis process are performed.

The RIS 30 is an order issuing device that issues and stores examination order information and transmits the issued examination order information to the movable radiography apparatus 10 via the communication network N.

The PACS 40 is an image management apparatus. To be specific, the PACS 40 stores and manages medical images (still images and dynamic images) generated by modalities such as the movable radiography apparatus 10 and analysis results obtained by the dynamic analysis apparatus 20 in association with patient information and examination information (for example, examination IDs, examination dates and times, imaging sites, and imaging conditions).

### (Operation)

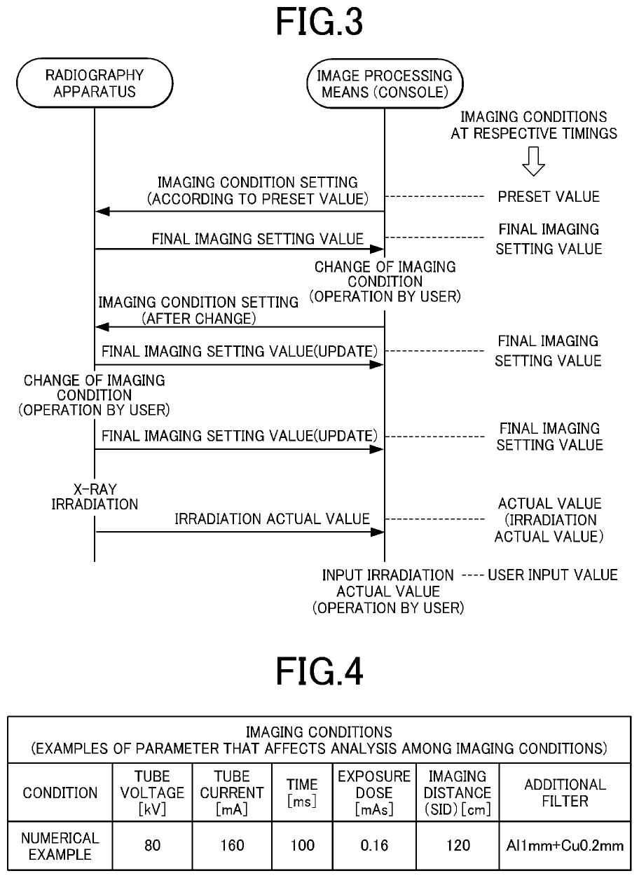

Next, referring to FIG. 3 to FIG. 8, a description will be provided on operation of the movable radiography apparatus 10 including the main body 1 as a dynamic image processing apparatus, operation (action) of the dynamic image processing system 100 including the movable radiography apparatus 10, and a dynamic image processing method.

FIGS. 5 to 7 are flowcharts illustrating one aspect of a dynamic image processing method. Note that in the flowcharts illustrated in FIGS. 5 to 7, the flow of signals is indicated by broken lines.

When the details of the order information are input (specified) by a doctor or the like and issuance of order information is instructed, the RIS 30 issues and transmits the order information to the movable radiography apparatus 10.

When the order information from the RIS 30 is received in the movable radiography apparatus 10, the controller 101 stores the received order information in the storage section 102. The controller 101 causes the display part 104 to display the order information on a display screen (not illustrated). As described above, the order information includes, for example, an examination ID, an examination date, patient information, information related to each imaging included in the examination, and the like.

When any piece of the order information is selected by an operation in the operation part 103 constituted by an operation button, a touch screen, or the like, a screen or the like for setting imaging conditions (irradiation conditions or image reading conditions) corresponding to each imaging order in the radiation sources 3 or the FPD 2 is displayed on the display part 104 or the like. Then, an input operation is performed on these screens, and thus imaging conditions are set.

Specifically, the controller 101 reads the imaging conditions corresponding to the order with reference to the table or the like of the storage section 102, and the controller 101 sets the irradiation conditions among the read imaging conditions in the drive section 108. The irradiation conditions include, for example, a tube voltage, a tube current, an irradiation time, an exposure dose, an imaging distance, grid information, and a frame rate. Furthermore, the controller 101 transmits image reading conditions (e.g., frame rate and pixel size) among the read imaging conditions to the FPD 2 via the FPD connection IF.

Note that a user may manually set the imaging conditions. In this case, in a case where there is another imaging of the same patient and the same part, the imaging condition that is once set may be automatically taken over without repeating the setting of the imaging condition. Thus, a work burden on the user can be reduced.

Usually, in the case of imaging in a general imaging room (not shown), a grid is mounted on an imaging table to which FPD 2 is set. When imaging is performed in a state in which the grid is present, scattered radiation is reduced, and thus the necessity of performing scattered ray removing process by image processing is low. In contrast, the movable radiography apparatus 10 such as a medical cart can be easily moved, but usually does not include equipment such as a grid. It is necessary to perform scattered ray removing process on the image captured in a state where the grid is not present in the dynamic image processing apparatus. The dynamic image processing apparatus is the main body 1 of the movable radiography apparatus 10 in the present embodiment. This similarly applies to a case where the imaging is the capture of a still image and a case where the imaging is the capture of a dynamic image.

Hereinafter, a case where the order is the dynamic imaging and the imaging is performed in a state where there is no grid will be described.

The dynamic image processing (scattered ray removing process) performed in the main body 1 of the movable radiography apparatus 10 as the dynamic image processing apparatus is performed by applying various parameters set based on imaging conditions and the like. The influence of scattered rays on a radiographic image varies depending on the body thickness of the subject S, setting conditions (imaging conditions) at the time of radiography, and the like.

Therefore, the body thickness of the subject S, imaging conditions, and the like are considered as parameters to be applied to the scattered ray removing process. Among these, parameters other than the imaging conditions, such as the body thickness of the subject S, are determined before imaging. Therefore, it is a numerical value that can be acquired on the main body 1 (dynamic image processing apparatus) side without waiting for the end of imaging.

On the other hand, the contents of the setting conditions (“imaging conditions”) set for radiography vary depending on the timing at which the main body 1 (the controller 101 of the main body 1) serving as the image processing unit (dynamic image processing apparatus) acquires the “imaging conditions”.

FIG. 3 is an explanatory view showing the contents exchanged between the radiography apparatus (radiation source 3) for irradiating the radiation X and the main body 1 as the image processing means (dynamic image processing apparatus, console) and the contents of the “imaging conditions” at each time point (timing).

As shown in FIG. 3, at the initial stage of the imaging instruction, the parameter(s) (“preset value(s)”) stored in the storage section 102 or the like are set as “imaging conditions” from the main body 1 serving as the console to the radiation source 3 serving as the radiography apparatus.

The radiography apparatus (radiation source 3) inquires of the console (main body 1) whether to perform actual imaging according to the received “imaging condition”. The “imaging condition” required to be checked is the “final imaging setting value” grasped by the console (main body 1) at this time (timing).

Thereafter, when the user operates the operation part 103 of the console (main body 1) or the like to input an instruction to change the “imaging condition”, an instruction to set the changed “imaging condition” is transmitted to the radiography apparatus (radiation source 3). The radiography apparatus (radiation source 3) updates the “imaging condition” to the changed one, and inquires of the console (main body 1) whether or not to actually perform imaging under the updated “imaging condition”.

When the user operates the radiography apparatus (radiation source 3) to input an instruction to change the “imaging condition”, the “imaging condition” is similarly updated to the changed one. Then, the radiography apparatus (radiation source 3) inquires of the console (main body 1) to check whether or not to perform actual imaging under the updated “imaging condition”.

In any case, the updated “imaging condition” for which the checking is requested becomes the “final imaging setting value” grasped by the console (main body 1) at this time (timing).

Then, in a case in which the radiation X is actually emitted in the radiography apparatus (radiation source 3), the console (main body 1) is notified of the setting conditions in a case in which imaging is actually performed as an “actual value” (irradiation actual value). The “imaging condition” grasped by the console (main body 1) at this time (timing) is the “actual value”.

In a case where the radiography apparatus (radiation source 3) does not cooperate with the console (main body 1), the console (main body 1) is not notified of the “actual value” at the time of the final actual imaging. In such a case, the user manually gives the console (main body 1) a value used for actual imaging by the radiography apparatus (radiation source 3). In this case, the “input value by the user” becomes the final “imaging condition” after imaging, instead of the “actual value”.

In this way, the “imaging condition” changes depending on the timing of acquisition by the console (main body 1). Then, depending on the timing of the “imaging condition” based on which the parameter used in the scattered ray removing process is set, a difference in the accuracy of the processing occurs. The accuracy of the scattered ray removing process affects the accuracy of the analysis process to be performed later on the dynamic image by the dynamic analysis apparatus 20.

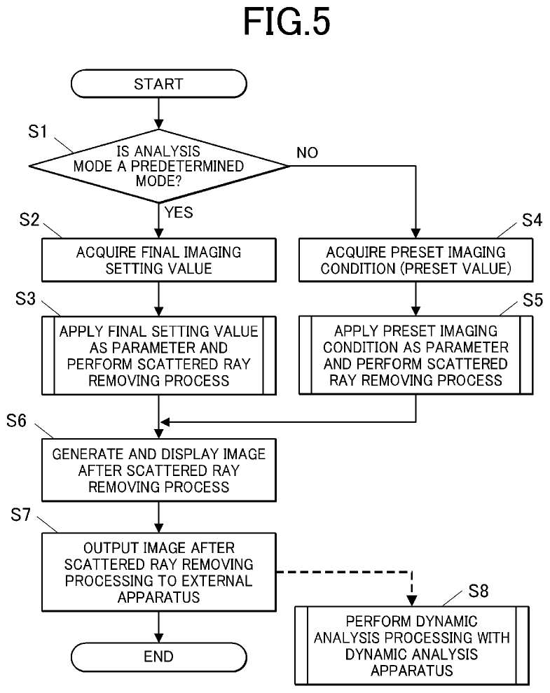

Among the imaging conditions, as shown in FIG. 4, for example, the tube voltage [kV], the tube current [mA], the irradiation time [ms], the exposure dose [mAs], the imaging distance (SID) [cm], the presence or absence of an additional filter, and the type of filter are parameters that particularly affect the analysis process (dynamic analysis).

In consideration of the accuracy of the analysis process (dynamic analysis) performed by the dynamic analysis apparatus 20, there is a demand for performing processing with high accuracy by applying parameters based on “imaging conditions” (i.e., actual values) in imaging actually performed as much as possible for the scattered ray removing process. However, the final “actual value” and the alternative “input value” are acquired post hoc after actual imaging is performed. Therefore, if the scattered ray removing process is performed after waiting for the acquisition of the “actual value” or the “input value”, the completion of the processing is delayed, and the patient is kept waiting for a long time.

Therefore, in the present embodiment, “imaging conditions” to be applied as parameters in the scattered ray removing process are changed according to the type of the analysis process (dynamic analysis) to be performed by the dynamic analysis apparatus 20. The type of analysis process is the type of analysis mode.

There are various methods of performing the scattered ray removing process by appropriately changing the “imaging condition” applied as a parameter. The method of performing the scattered ray removing process may be selected by the user. Each of the methods will be described below.

First, in the first method, as illustrated in FIG. 5, the controller 101 of the main body 1 serving as the dynamic image processing apparatus determines whether or not the analysis mode (type of analysis) of the analysis process (dynamic analysis) performed by the dynamic analysis apparatus 20 is a predetermined mode (predetermined analysis process) (step S1). As described above, the “predetermined analysis process” is, for example, a blood flow analysis process or a ventilation analysis process in the case of a dynamic image obtained by imaging the chest of the subject. Note that a process other than these may be included in the “predetermined analysis processes” as long as it is a process for which it is necessary to analyze a minute change or difference in signal value. The analysis mode of the dynamic analysis is included in, for example, the order information (information on the examination order) provided from the RIS 30. The controller 101 determines the analysis mode of the dynamic analysis to be performed on the dynamic image based on the examination order.

When the analysis mode of the dynamic analysis is the predetermined mode (predetermined analysis process) (step S1; YES), the controller 101 acquires the final imaging setting values among the “imaging conditions” set for the imaging of the dynamic image (step S2).

Next, the controller 101 applies the final imaging setting value as a parameter and performs the scattered ray removing process (step S3). Note that in this case, the controller 101 may acquire an actual value rather than the final imaging setting value after the end of imaging and perform the scattered ray removing process using the actual value.

On the other hand, in a case where the analysis mode of the dynamic analysis is not the predetermined mode (predetermined analysis process) (step S1; NO), it is not necessary to withstand the highly accurate analysis process. However, processing for displaying an image having a certain degree of sharpness on the display part 104 or the like of the main body 1 is performed. That is, in this case, the controller 101 acquires the preset value among the “imaging conditions” set for capturing the dynamic image (step S4).

Next, the controller 101 applies the preset value as the parameter and performs the scattered ray removing process (step S5). In this case, the scattered ray removing process can be performed without waiting for the notification of the final imaging setting value or the actual value. Therefore, the scattered ray removing process for a dynamic image that requires a relatively long time can be started early, and the waiting time can be reduced.

Next, regardless of which process has been performed, an image after the scattered ray removing process is generated and displayed on the display part 104 or the like (step S6). Then, the image after the scattered ray removing process is output to an external device such as the dynamic analysis apparatus 20 (step S7).

The dynamic analysis apparatus 20 that has received the image after the scattered ray removing process appropriately performs the dynamic analysis process on the image after the scattered ray removing process (step S8).

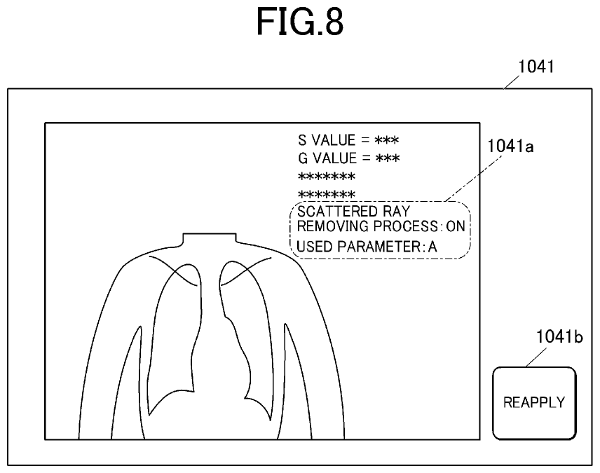

Note that only various values and the like may be displayed on a display screen 1041 (see FIG. 8) of an image after the scattered ray removing process displayed on the display part 104 of the main body 1, a display unit (not shown) of the dynamic analysis apparatus 20, or the like. On the display part 104 or the like, it is preferable that a display field 1041a of information regarding the scattered ray removing process regarding the image after the scattered ray removing process displayed on the display screen 1041 is overlaid and displayed.

In the example illustrated in FIG. 8, the presence or absence of the scattered ray removing process and the type of “imaging condition” applied as a parameter in the scattered ray removing process are displayed in a display field 1041a as the information related to the scattered ray removal. As for the presence or absence of the scattered ray removing process, for example, in the case of an image to which the scattered ray removing process has already been applied, “scattered ray removing process: ON” is displayed as shown in FIG. 8. Further, in a case where the scattered ray removing process has not been applied, “scattered ray removing process: OFF” is displayed.

For example, in a case where the used parameter is the final imaging setting value, “used parameter: A” is displayed. Furthermore, in a case where the used parameter is an actual value, “used parameter: R” is displayed. Furthermore, in a case where the used parameter is a preset value, (no mark) is displayed in the field of “used parameter:”. In this way, the user can easily identify whether or not the image displayed on the display screen has been subjected to the scattered ray removing process, and which imaging condition has been applied as a parameter in a case where the image has been subjected to the process.

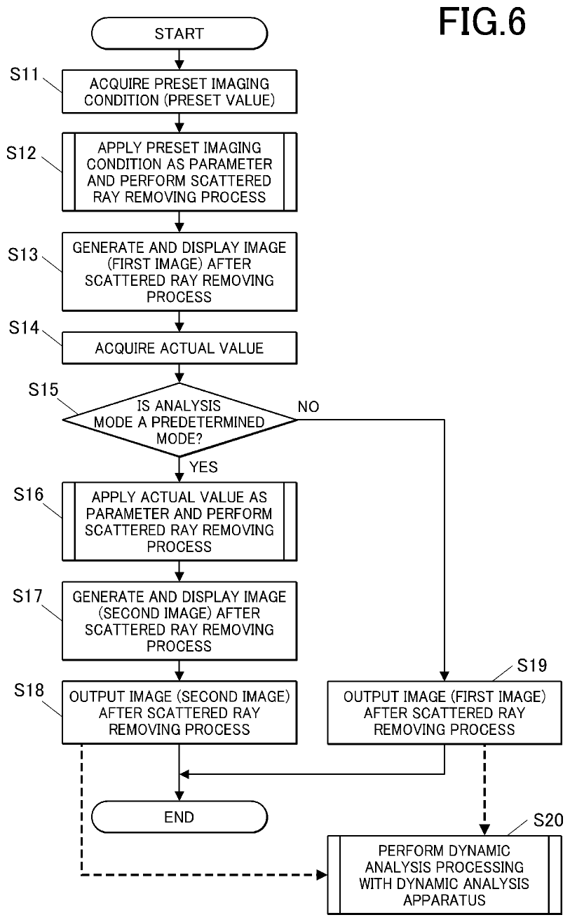

Furthermore, according to the second method, as illustrated in FIG. 6, the controller 101 of the main body 1 acquires a preset value among the “imaging conditions” set for capturing a dynamic image (step S11). Then, the controller 101 performs the scattered ray removing process by applying the preset value as a parameter (step S12). In this case, the scattered ray removing process can be performed without waiting for the notification of the final imaging setting value or the actual value. Therefore, it is possible to quickly start the scattered ray removing process for the dynamic image which requires a relatively long time.

Next, an image (first image) after the scattered ray removing process is generated and is displayed on the display part 104 or the like (step S13).

Then, in a case where radiography is actually performed, the actual value which is the value actually used in imaging is notified from the radiation source 3 which is the radiography apparatus to the controller 101 of the main body 1 after the end of imaging (step S14).

Then, the controller 101 of the main body 1 determines whether the analysis mode (type of analysis) of the analysis process (dynamic analysis) performed by the dynamic analysis apparatus 20 is a predetermined mode (predetermined analysis process) (step S15).

When the analysis mode of the dynamic analysis is the predetermined mode (predetermined analysis process) (step S15; YES), the controller 101 performs the scattered ray removing process by applying the actual value as a parameter (step S16). Note that in this case, the controller 101 may acquire the final imaging setting value rather than the actual value and perform the scattered ray removing process using the final imaging setting value. However, in the case of this method 2, an image (first image) after the scattered ray removing process in which the preset value is applied as a parameter has already been acquired. Therefore, it is less necessary to perform processing in a hurry. Therefore, when a final actual value has been obtained, it is preferable to perform the scattered ray removing process by applying this value.

Next, the controller 101 generates an image (second image) after the scattered ray removing process and causes the display part 104 or the like to display the image (step S17). Furthermore, the image (second image) after the scattered ray removing process is output to an external device such as the dynamic analysis apparatus 20 (step S18).

On the other hand, when the analysis mode of the dynamic analysis is not the predetermined mode (predetermined analysis process) (step S15; NO), the controller 101 applies the preset value as a parameter to perform the scattered ray removing process and outputs the image after the scattered ray removing process (first image) to an external device such as the dynamic analysis apparatus 20 (step S19).

Note that the analysis mode (type of analysis) of the analysis process (dynamic analysis) is determined. Therefore, the scattered ray removing process may be performed by limiting the frame range according to the analysis mode. For example, targets to be processed are narrowed down to an analysis range or an output range such that processing is limited to a range of a respiratory cycle only used for analysis in a case of ventilation analysis. This leads to a reduction in processing load and analysis time. Alternatively, the range may be limited only in a case of the image (first image) after the scattered ray removing process using a preset value or the like.

The dynamic analysis apparatus 20 that has received the image (the first image or the second image) after the scattered ray removing process performs the dynamic analysis process as appropriate on the image after the scattered ray removing process (step S20).

It is preferable that a display field 1041a of information regarding the scattered ray removing process regarding the displayed image (the first image or the second image) after the scattered ray removing process is displayed on the display screen 1041 (refer to FIG. 8) of the image after the scattered ray removing process displayed on the display part 104 of the main body 1, the display part of the dynamic analysis apparatus 20, or the like. This point is the same as the case of the first method.

The third method is a flowchart assuming a configuration in which the radiography apparatus and the image processing apparatus (console) do not cooperate with each other.

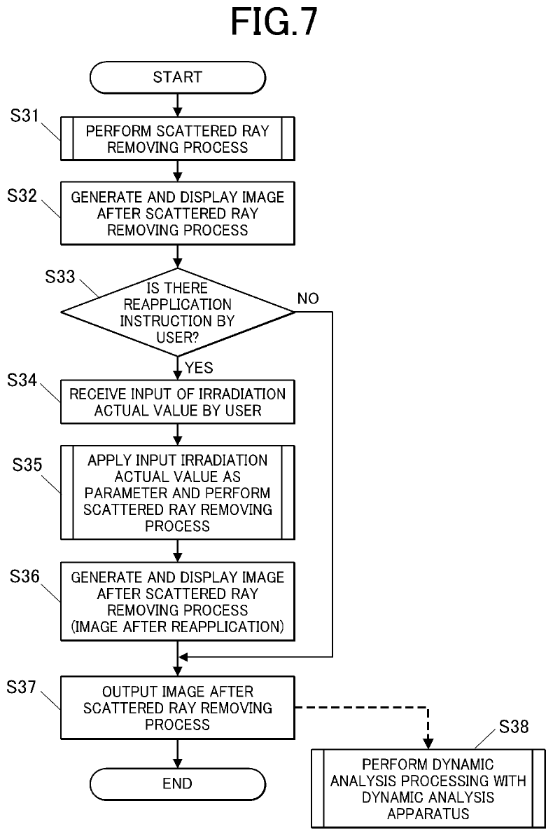

In this case, as illustrated in FIG. 7, the controller 101 of the main body 1 performs the scattered ray removing process by applying various parameters (step S31). Next, the controller 101 generates an image after the scattered ray removing process and causes the display part 104 or the like to display the image (step S32). As described above, in a case where the radiography apparatus and the image processing apparatus (console) do not cooperate with each other, it is not assumed that the actual value or the like actually used for imaging is notified from the radiography apparatus. For this reason, a preset value or the like stored in the storage section 102 or the like of the main body 1 which is the image processing apparatus (console) is applied as a parameter in a case in which the scattered ray removing process is performed.

Next, the controller 101 determines whether or not an input instructing to reapply the scattered ray removing process has been made by the user (step S33).

As illustrated in FIG. 8, a reapplication button 1041b for inputting whether or not to reapply the scattered ray removing process to the image is provided on the display screen 1041.

The user views the image displayed on the display screen 1041 and operates the reapplication button 1041b in a case where the scattered ray removing process is insufficient or the like. When the reapplication button 1041b is operated by the user, the controller 101 determines that an input instructing to reapply the scattered ray removing process has been made by the user (step S33; YES).

Also in the third method, on the display screen 1041 (refer to FIG. 8) of the image after the scattered ray removing process displayed on the display part 104 or the like of the main body 1, the image after the scattered ray removing process is displayed, and the display field 1041a of the information regarding the scattered ray removing process applied to the image is displayed. This is the same as the case of the first method and the case of the second method.

The information displayed in the display field 1041a is, for example, what stage of “imaging condition” has been used as a parameter to perform the scattered ray removing process. That is, this information indicates which of the preset value, the final imaging setting value, and the actual value is set as the parameter. A user can easily check these pieces of information from the screen, and can use them as materials for determining whether or not to reapply the scattered ray removing process.

In a case in which the reapplication button 1041b is operated by the user, the user manually inputs the irradiation actual value at the time of imaging from the operation part 103 or the like of the main body 1, and the controller 101 receives the input actual value (step S34).

When input of an actual value (irradiation actual value) is accepted, the controller 101 as an image processing means applies the actual value as a parameter and performs the scattered ray removing process (step S35).

Then, an image after the scattered ray removing process (an image after the reapplication) is generated and displayed on the display part 104 or the like (step S36). In addition, the image after the scattered ray removing process (image after reapplication) is output to an external device such as the dynamic analysis apparatus 20 (step S37).

On the other hand, when an instruction to reapply the scattered ray removing process is not input by the user (step S33; NO), the image after the scattered ray removing process generated in step S32 is output to an external device such as the dynamic analysis apparatus 20 (step S37). Note that in response to the output (or at the timing of the output), a confirmation display may be made on the display part 104 or the like to confirm whether or not the scattered ray removed image with the preset value may be output in a case where the scattered ray removing process with the irradiation actual value can be reapplied. Providing such a confirmation display can draw the user's attention.

The dynamic analysis apparatus 20 that has received the image after the scattered ray removing process (the image generated in Step S32 or the image after reapplication generated in Step S36) appropriately performs the dynamic analysis process on the image after the scattered ray removing process (Step S38).

In the case of the third method, although it depends on determination by the user, in a case where the image after the scattered ray removing process does not have quality suitable for the subsequent dynamic analysis process in the dynamic analysis apparatus 20, the scattered ray removing process is reapplied using the value input by the user instead of the actual value as a parameter. Therefore, the dynamic analysis process is not performed on an image with poor quality, and it is possible to prevent a decrease in the quality of the dynamic analysis.

### Effect

As described above, according to the main body 1 of the movable radiography apparatus 10 as the dynamic image processing apparatus, the controller 101 executes the scattered ray removing process on the dynamic image captured by irradiating the subject S with the radiation X. Further, the controller 101 acquires at least one of “imaging conditions” set for capturing a dynamic image. Then, the controller 101 performs the scattered ray removing process on the dynamic image based on at least one of the “predetermined condition” and the “imaging condition” acquired as the acquisition means.

Even when the dynamic analysis is performed on an image on which the scattered ray removing process is not appropriately performed, a precise analysis result may not be obtained. In this regard, in the present embodiment, the scattered ray removing process can be appropriately performed on the dynamic image. For this reason, it is possible to generate an image suitable for a dynamic image and to perform favorable dynamic analysis.

In the present embodiment, at least one of the “imaging conditions” is a condition (irradiation actual value) actually used in the imaging of the dynamic image.

Therefore, it is possible to perform appropriate scattered ray removing process based on actual imaging conditions. For this reason, it is possible to generate an image suitable for a dynamic image and to perform favorable dynamic analysis.

In the present embodiment, at least one of the “imaging conditions” is the final imaging setting value set for capturing the dynamic image.

As illustrated in FIG. 3, the final imaging setting value may be changed and updated until the imaging is actually executed. However, the final imaging setting value is a value relatively close to the actual value (irradiation actual value). Therefore, it is possible to perform appropriate scattered ray removing process that is more suited to actual imaging conditions than in using a preset value as a parameter. For this reason, it is possible to generate an image suitable for a dynamic image and to perform favorable dynamic analysis.

Furthermore, in the present embodiment, the storage section 102 is included as a storage means that stores predetermined parameters (preset values) for the scattered ray removing process. Next, the controller 101 acquires at least one of the conditions (irradiation actual value) actually used in the capturing of the dynamic image or the final imaging setting value set for the capturing of the dynamic image. Furthermore, the controller 101 performs the scattered ray removing process on the dynamic image on the basis of the parameters (preset values) stored in the storage section 102 to generate a “first scattered ray removed image”. In addition, in a case where the “predetermined condition” is satisfied, the controller 101 further performs the scattered ray removing process on the dynamic image based on the conditions (i.e., the irradiation actual value and the final imaging setting value) acquired as the acquisition means and generates the “second scattered ray removed image”.

In a case where the “first scattered ray removed image” is generated, processing can be performed without waiting for the actual value or the like, and the waiting time of the patient can be reduced. Furthermore, even if it takes some time to generate the “second scattered ray removed image”, it is possible to provide the dynamic analysis apparatus 20 with an image that can withstand highly accurate dynamic analysis by performing appropriate scattered ray removing process in accordance with actual imaging conditions.

Therefore, flexible measures can be taken depending on the accuracy and the like required for the dynamic analysis.

Further, in the present embodiment, the “case corresponding to the predetermined condition” is a case where the type of the analysis process performed after the scattered ray removing process includes the “predetermined analysis process”, and where the examination order or the like includes the “predetermined analysis process”. Note that whether or not to perform the “predetermined analysis process” may be included in the examination order, or may be known by checking condition settings in the main body 1 serving as the image processing apparatus (console).

As a result, even in a case where the image (dynamic image) related to the scattered ray removing process has high accuracy that requires determination of a slight difference in signal value or the like, it is possible to provide the dynamic analysis apparatus 20 with an image that can withstand highly accurate dynamic analysis.

Therefore, flexible measures can be taken depending on the accuracy and the like required for the dynamic analysis.

In the present embodiment, the type of the “predetermined analysis process” is, for example, at least one of a blood flow analysis process and a ventilation analysis process.

When the blood flow analysis process and the ventilation analysis process are performed by the dynamic analysis, it is necessary to perform a highly accurate analysis without missing a slight change in signal value or the like. In this regard, in the present embodiment, it is possible to generate an image that can withstand such highly accurate dynamic analysis and provide the image to the dynamic analysis apparatus 20.

Therefore, favorable dynamic analysis can be performed.

The “case corresponding to a predetermined condition” may be a case where an input by the operator for reapplication of the scattered ray removing process is received.

In this case, although it depends on the user's determination, in a case where the image after the scattered ray removing process is not of a quality suitable for the subsequent dynamic analysis process in the dynamic analysis apparatus 20, the scattered ray removing process can be applied again using a value input by a user or the like as a parameter instead of the actual value. Therefore, the dynamic analysis process is not performed on an image with poor quality, and it is possible to prevent a decrease in the quality of the dynamic analysis.

In addition, in a case where the “first dynamic analysis process” is performed on the dynamic image, after the “first scattered ray removed image” is generated, an image based on a “second scattered ray removed image” generated by performing the scattered ray removing process based on at least one of the “imaging conditions” acquired by the controller 101 as the acquisition means is output. In addition, in a case where the “second dynamic analysis process” is performed on the dynamic image, an image based on the “first scattered ray removed image” is output.

When the “first scattered ray removed image” is generated, processing can be performed without waiting for the actual value or the like, and a waiting time for a patient can be reduced, and when the “second scattered ray removed image” is generated, even if it takes some time, an appropriate scattered ray removing process corresponding to actual imaging conditions is performed, and the dynamic analysis apparatus 20 can be provided with an image which can withstand highly accurate dynamic analysis.

Therefore, flexible measures can be taken depending on the accuracy and the like required for the dynamic analysis.

Further, the controller 101 of the main body 1 as the dynamic image processing apparatus executes the scattered ray removing process on the dynamic image captured by irradiating the subject S with the radiation X. Furthermore, the controller 101 acquires at least one of “imaging conditions” set for capturing of a dynamic image. The storage section 102 of the main body 1 as the dynamic image processing apparatus stores a predetermined parameter (preset value) for the scattered ray removing process. The controller 101 performs scattered ray removing process on the dynamic image on the basis of parameters (preset values and the like) to generate a “first scattered ray removed image”. Next, the controller 101 performs the scattered ray removing process on the dynamic image on the basis of at least one of the acquired “imaging conditions” to generate a “second scattered ray removed image”.

When the “first scattered ray removed image” is generated, processing can be performed without waiting for an actual value or the like, and the waiting time of the patient can be reduced. Furthermore, even if it takes some time to generate the “second scattered ray removed image”, it is possible to provide the dynamic analysis apparatus 20 with an image that can withstand highly accurate dynamic analysis by the appropriate scattered ray removing process in accordance with actual imaging conditions.

Therefore, flexible measures can be taken depending on the accuracy and the like required for the dynamic analysis.

Furthermore, in the present embodiment, the main body 1 which is the dynamic image processing apparatus is mounted on a movable radiography apparatus such as a so-called medical cart.

Accordingly, even in a case where it is difficult to perform radiography using a grid in an imaging room or the like, such as a case where a patient who cannot freely move his/her body is the subject S, the scattered ray removing process can be appropriately performed on the dynamic image. For this reason, it is possible to generate an image suitable for a dynamic image and to perform favorable dynamic analysis.

### Modification Example

Note that the description in the above embodiment is a preferred example of the present invention, and the present invention is not limited to this.

For example, although the case where the dynamic image processing apparatus of the present invention is applied to a movable radiography apparatus (a so-called medical cart or the like) has been described as an example in the above embodiment, the present invention is not limited to the case where it is applied to the movable radiography apparatus.

For example, the dynamic image processing apparatus of the present invention may be applied to a console or the like of a stationary radiography apparatus disposed in a normal imaging room or the like. Even in the case of a stationary radiography apparatus, it is assumed that imaging is performed without using a grid when, for example, imaging is performed in a state in which only the FPD is taken out from the apparatus main body and disposed under the subject S. In particular, in such a case, the scattered ray removing process by the dynamic image processing apparatus of the present invention is useful.

Further, in the above description, an example in which a hard disk, a semiconductor nonvolatile memory, or the like is used as a computer-readable medium of the program according to the present invention has been disclosed, but the present invention is not limited to this example. As other computer-readable media, portable recording media such as CD-ROMs can be applied. Furthermore, a carrier wave is also applied as a medium for providing data of the program according to the present invention via a communication line.

In addition, the detailed configuration and the detailed operation of each apparatus constituting the dynamic image processing apparatus, the movable radiography apparatus, and the dynamic image processing system can be appropriately changed without departing from the scope of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. A dynamic image processing apparatus comprising:
a hardware processor that
performs a scattered ray removing process on a dynamic image captured by irradiating a subject with radiation; and
acquires at least one imaging condition that is set in capturing the dynamic image;
wherein the hardware processor performs the scattered ray removing process on the dynamic image based on at least one of a predetermined condition and the imaging condition that has been acquired.

2. The dynamic image processing apparatus according to claim 1,
wherein the at least one imaging condition is a condition that has been actually used in capturing the dynamic image.

3. The dynamic image processing apparatus according to claim 1,
wherein the at least one imaging condition is a final setting condition that has been set in capturing the dynamic image.

4. The dynamic image processing apparatus according to claim 1, further comprising: a storage that stores a predetermined parameter used in the scattered ray removing process;
wherein the hardware processor acquires at least one of a condition that has been actually used in capturing the dynamic image and a final setting condition that has been set in capturing the dynamic image,
wherein the hardware processor performs the scattered ray removing process on the dynamic image based on the parameter and generates a first scattered ray removed image, and
wherein, upon the predetermined condition being satisfied, the hardware processor further performs the scattered ray removing process on the dynamic image based on the condition that has been acquired by the hardware processor and generates a second scattered ray removed image.

5. The dynamic image processing apparatus according to claim 1,
wherein the predetermined condition is that an analysis process to be performed after the scattered ray removing process includes a predetermined analysis process.

6. The dynamic image processing apparatus according to claim 5,
wherein the predetermined condition is that an examination order includes the predetermined analysis process.

7. The dynamic image processing apparatus according to claim 5,
wherein the predetermined analysis process includes at least one of a blood flow analysis process and a ventilation analysis process.

8. The dynamic image processing apparatus according to claim 1,
wherein the predetermined condition is that an input by an operator for reapplication of the scattered ray removing process has been received.

9. The dynamic image processing apparatus according to claim 5,
wherein, upon the analysis process being a first dynamic analysis process performed on the dynamic image, an image based on a second scattered ray removed image is output, the second scattered ray removed image being generated by the scattered ray removing process based on the at least one imaging condition after generation of a first scattered ray removed image, and
wherein, upon the analysis process being a second dynamic analysis process performed on the dynamic image, an image based on the first scattered ray removed image is output.

10. A dynamic image processing apparatus comprising:
a hardware processor that
performs a scattered ray removing process on a dynamic image captured by irradiating a subject with radiation; and
acquires at least one imaging condition that is set for capturing the dynamic image; and
a storage that stores a predetermined parameter used in the scattered ray removing process;
wherein the hardware processor performs a scattered ray removing process on the dynamic image based on the parameter to generate a first scattered ray removed image, and the scattered ray removing process on the dynamic image based on at least one imaging condition that has been acquired by the hardware processor to generate a second scattered ray removed image.

11. A movable radiography apparatus comprising the dynamic image processing apparatus according to claim 1.

12. A dynamic image processing system comprising:
a radiation emitting apparatus that emits radiation;
a radiography apparatus that generates a dynamic image based on radiation emitted from the radiation emitting apparatus and transmitted through a subject;
a dynamic image processing apparatus that performs scattered ray removing process on the dynamic image; and
an analysis processing apparatus that performs a dynamic analysis process on the dynamic image on which the scattered ray removing process has been performed,
wherein the dynamic image processing apparatus performs the scattered ray removing process based on at least one of a predetermined condition and an imaging condition that is set for capturing the dynamic image.

13. The dynamic image processing system according to claim 12,
wherein the at least one imaging condition is a condition that has been actually used in capturing the dynamic image.

14. The dynamic image processing system according to claim 12,
wherein the at least one imaging condition is a final setting condition that has been set in capturing the dynamic image.

15. The dynamic image processing system according to claim 12, further comprising: a storage that stores a predetermined parameter used in the scattered ray removing process;
wherein the dynamic image processing apparatus acquires at least one of a condition that has been actually used in capturing the dynamic image and a final setting condition that has been set in capturing the dynamic image,
wherein the dynamic image processing apparatus performs the scattered ray removing process on the dynamic image based on the parameter to generate a first scattered ray removed image, and
wherein, upon the predetermined condition being satisfied, the dynamic image processing apparatus further performs the scattered ray removing process on the dynamic image based on the condition acquired by the dynamic image processing apparatus to generate a second scattered ray removed image.

16. The dynamic image processing system according to claim 12,
wherein the predetermined condition is that an analysis mode of a dynamic analysis process by the analysis processing apparatus includes a predetermined mode.

17. The dynamic image processing system according to claim 16,
wherein the predetermined condition is that an examination order includes the predetermined mode.

18. The dynamic image processing system according to claim 16,
wherein, upon the dynamic analysis process being a first dynamic analysis process performed on the dynamic image, the analysis processing apparatus performs the dynamic analysis process on an image based on a second scattered ray removed image is output, the second scattered ray removed image being generated by the scattered ray removing process based on the at least one imaging condition that has been acquired by the dynamic image processing apparatus after generation of a first scattered ray removed image, and
wherein, upon the dynamic analysis process being a second dynamic analysis process performed on the dynamic image, the analysis processing apparatus performs the dynamic analysis process on the first scattered ray removed image.

19. The dynamic image processing system according to claim 16,
wherein the predetermined mode is at least one of a blood flow analysis process and a ventilation analysis process.

20. The dynamic image processing system according to claim 12,
wherein the predetermined condition is that an input for reapplication of the scattered ray removing process by an operator has been received.

21. A dynamic image processing system comprising:
a radiation emitting apparatus that emits radiation;
a radiography apparatus that generates a dynamic image based on radiation emitted from the radiation emitting apparatus and transmitted through a subject;
a dynamic image processing apparatus that performs scattered ray removing process on the dynamic image;
a storage that stores a predetermined parameter used in the scattered ray removing process; and
an analysis processing apparatus that performs a dynamic analysis process on the dynamic image on which the scattered ray removing process has been performed,
wherein the dynamic image processing apparatus performs a first scattered ray removing process on the dynamic image based on the parameter and performs a second scattered ray removing process on the dynamic image based on at least one of a predetermined condition and an imaging condition that is set for capturing the dynamic image.

22. A computer-readable recording medium storing a program which, when executed by a hardware processor, causing the hardware processor to perform:
an image processing function of performing a scattered ray removing process on a dynamic image captured by irradiating a subject with radiation; and
an acquisition function of acquiring at least one imaging condition that is set for capturing the dynamic image,
wherein, in the image processing function, the hardware processor performs the scattered ray removing process on the dynamic image based on at least one of a predetermined condition and the imaging condition that has been acquired by the acquisition function.

23. A dynamic image processing method comprising:
image processing of performing a scattered ray removing process on a dynamic image that has been captured by irradiating a subject with radiation; and
acquiring at least one imaging condition that is set for capturing the dynamic image,
wherein, in the image processing, the scattered ray removing process is performed on the dynamic image based on at least one of a predetermined condition and the imaging condition acquired in the acquiring.
